(19) United States
(12) Patent Application Publication
(10) Pub. No.: US 2007/0185009 A1
(43) Pub. Date: Aug. 9, 2007

(54) Coated catheters

(75) Inventor: Evan C. Unger, Tucson, AZ (US)

Correspondence Address: DLA PIPER US LLP, 4365 EXECUTIVE DRIVE, SUITE 1100, SAN DIEGO, CA, 92121-2133, US

(73) Assignee: IMARX THERAPEUTICS, INC., Tucson, AZ (US)

(21) Appl. No.: 11/655,620

(22) Filed: Jan. 19, 2007

Related U.S. Application Data
(60) Provisional application No. 60/760,477, filed on Jan. 20, 2006.

(51) Int. Cl. A61K 38/00

(52) U.S. Cl. 514/2

(57) ABSTRACT

Provided herein are coated medical devices. More specifically, at least a portion of the device (e.g., catheter) is coated with an active component, wherein the active component prevents or treats occlusions and the formation(s) thereof.

### CROSS-REFERENCE TO RELATED APPLICATIONS

This application claims priority under 35 U.S.C. §119(e) to U.S. Patent Application Ser. No. 60/760,477, filed Jan. 20, 2006, the entire contents of which is incorporated herein by reference.

### BACKGROUND

1. Field of the Invention

The present invention relates to coated medical devices. More specifically, it relates to medical devices such as catheters, where at least a portion of the device is coated with an active component, wherein the active component prevents or treats occlusions and the formation thereof.

2. Background Information

Despite the widespread use of a variety of medical devices, such as catheters, there are several complications that may arise from their use. One such complication includes the formation of a thrombus which may occlude fluid and/or blood flow. Various strategies have been used in attempts to prevent the formation catheter thrombosis and occlusion from occurring, including, but not limited to, the use of heparin. For example, a continuous infusion of an anticoagulant such as heparin was used, which acts by binding to anti-thrombin III and inhibiting thrombogenesis primarily through inactivation of factors Ia and Xa. Despite using heparin in the above described manner, occlusions due to thrombus formation are still common.

The formation of a thrombus results in a time consuming clearing process. Upon noticing an occlusion, a health care provider must take immediate measures to remove the occlusion. Such measures include, but are not limited to, either flushing the catheter with saline and/or heparin to attempt to clear the occlusion. Again, this process is time consuming and potentially uncomfortable for the patient. Moreover, a caregiver must continually monitor the catheter for occlusions. This results in wasted caregiver time and overall increased healthcare expenses. Accordingly, there is a need to have better procedures preventing the formation of thrombi.

### SUMMARY

According to one embodiment of the present invention, a coated medical device is provided for reducing the incidence of, and/or prevents occlusions from, forming on the device, and/or caused by the presence of the device, the device comprising a body, at least one active component selected from a group consisting of a drug, a peptide, a ligand, a protein, and an antibody, and at least one bi-functional poly(alkylene glycol) disposed between the body and the active component, wherein the bi-functional poly(alkylene glycol) is bound at one end to the body and bound at the other end to the active component.

According to another embodiment of the present invention, such a device contains at least one carbon-containing component, at least one fluorinated component disposed between the body and the carbon-containing component, at least one active group, the active group being a plasminogen activator and/or a fibrinolytic enzyme, and at least one head group containing one or more phosphate groups disposed between the active group and the carbon-containing component.

### BRIEF DESCRIPTION OF THE DRAWINGS

FIG. 1 is a cross-sectional view showing a polyethylene glycol (PEG) group disposed between an exterior surface of a device and at least one active component.

FIG. 2 illustrates a cross-sectional view showing one embodiment of a coated device having a polyethylene glycol (PEG) group disposed between the device exterior surface and at least one active component.

FIG. 3 illustrates a cross-sectional view showing another embodiment of a coated device having a fluorinated linker and an active component adjacent thereto according to one aspect of the present invention.

FIG. 4 is a cut away perspective view of the coated device, without a PEG linker dispose between the head group component and the active component according to one aspect of the present invention.

FIG. 5 is a cut away perspective view of the coated device, wherein more than one fluorinated linker and at least one carbon-containing group is utilized according to one aspect of the present invention.

FIG. 6 is a cut away perspective view of the coated device, without a PEG linker dispose between the head group component and the active component and wherein more than one fluorinated linker and at least one carbon-containing group is utilized according to one aspect of the present invention.

### DETAILED DESCRIPTION

1. Terms and Definitions

The term “catheter” is defined to refer to a thin and flexible tube inserted into a bodily passage or cavity in order to allow fluids to pass into or out of it, to distend (expand) it, or to convey diagnostic or other instruments through it. Moreover, catheters may be made from many known materials via any industrially acceptable method(s).

The term “stent” is defined to refer to a tube designed to be inserted into a vessel or passageway to keep it open.

The term “guidewire” is defined to refer to a flexible wire positioned in an organ, vessel, or duct for the purpose of directing the passage of a larger device threaded over or along its length.

The term “balloon catheter” is defined to refer to a device used to open a narrowed artery or organ that has become blocked, the device including a hollow tube with a small, inflatable balloon at the tip.

The term “occlusion” is defined to include blood clots, thrombus, and any other formation and/or material that would preclude and/or prevent the flow of fluid.

The term “poly(alkylene glycol)” is defined to include any polymer derived from a monomer that comprises at least two hydroxyl groups separated by at least two methylene groups.

The terms “poly(ethylene glycol)” and “PEG,” also known as poly(ethylene oxide), refer to a polymer that is a species of a poly(alkylene glycol) and has the general structure HO—[CH₂—CH₂—O]ₙ—H.

The term “poly(propylene glycol),” also known as poly(propylene oxide), refers to a polymer that is a species of a poly(alkylene glycol) and has the general structure HO—CH(CH₃)[CH₂OCH(CH₃)]ₙ—CH₂OH.

The term “bi-functional activity,” as related to a poly(alkylene glycol), is defined to refer to the capacity of a poly(alkylene glycol) to bind to at least two different materials.

2. Embodiments of the Invention

The following detailed description of the invention is made with reference to the accompanying drawings which form a part hereof, and in which are shown, by way of illustration, specific embodiments in which the invention may be practiced. These embodiments are described in sufficient detail to enable those skilled in the art to practice the invention. Other embodiments may be utilized and changes may be made without departing from the scope of the present invention.

The present invention relates to coated medical devices. More specifically, the devices are coated with an active component to reduce the incidence of and/or to prevent occlusions from forming on, or due to the presence, of the device. The active component may be applied to, attached to, or otherwise made spatially adjacent to the surface of the catheter via a bond, linkage and/or other attachment to a poly(alkylene glycol) moiety

Any known poly(alkylene moiety) having at least bi-functional activity may be used in the present invention. One example of a poly(alkylene glycol) moiety that can be utilized is a poly(ethylene glycol) (PEG) moiety. Those having ordinary skill in the art can employ an alternative poly(alkylene glycol) moiety, if desired, for example, a poly(propylene glycol) moiety.

If PEG is to be selected, any PEG having the molecular weight less than about 25,000 Daltons can be used, such as the PEG having the molecular weight of between about 2,000 to about 10,000 Daltons, for example, PEG 3000, PEG 4000 and/or PEG 5000.

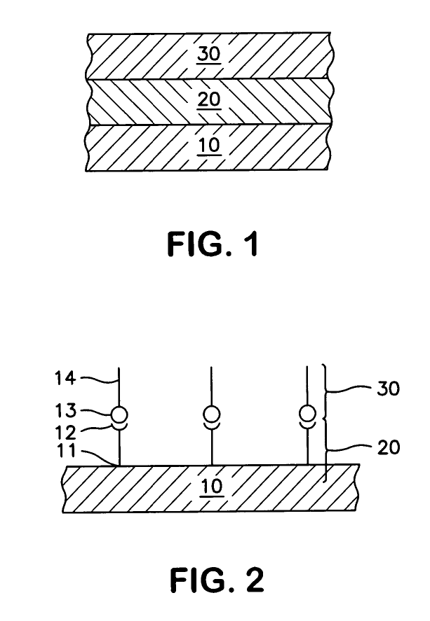

FIG. 1 illustrates a cross-sectional view of a coated device showing a PEG moiety 20 disposed between a surface or wall 10 of the device (e.g., a catheter wall) and at least one active component 30.

The poly(alkylene moiety), such as the PEG moiety 20, may be comprised of one or several PEG groups. For example, the bi-functional PEG group may bind at one end to a device surface or wall 10 comprised of polytetrafluoroethylene (PTFE) and an active component 30 bound, linked or otherwise attached to its other end.

The active component 30 may include a drug such as a plasminogen activator and/or fibrinolytic enzyme. Such drugs may include, but are not limited to, tissue plasminogen activator, urokinase, prourokinase, and/or fibrinolytic enzymes such as desmoteplase, alfimaprase, and/or ancrod. Also, the active component may be a peptide, ligand, protein, antibody, etc. The active component may also be any combination and/or derivation of any of the above.

The device may be any device that requires a coating on a surface to reduce the incidence of and/or to prevent occlusions from forming on or because of the device. Example of devices that can be used include a catheter, stent, guidewire or balloon catheter which may be inserted into a bodily passage or cavity in order to allow fluids to pass into or out of it, to distend (expand) it, or to convey diagnostic or other instruments through it. The device may be made from any physiologically acceptable materials, including, but not limited to, silicone, polyurethane, a fluorinated polymer such as polytetrafluoroethylene (PTFE), etc., or from any combination and/or derivations of any of the above materials.

Referring now to FIG. 2, the PEG moiety 20 includes two ends, with one end 11 attached to the device wall 10 and an opposite receiving end 12 bound with the active component 30. The active component 30 includes two ends, 13, and 14. End 13 is bound to receiving end 12 of the PEG moiety 20, leaving end 14 free to act via its specific mechanism of action.

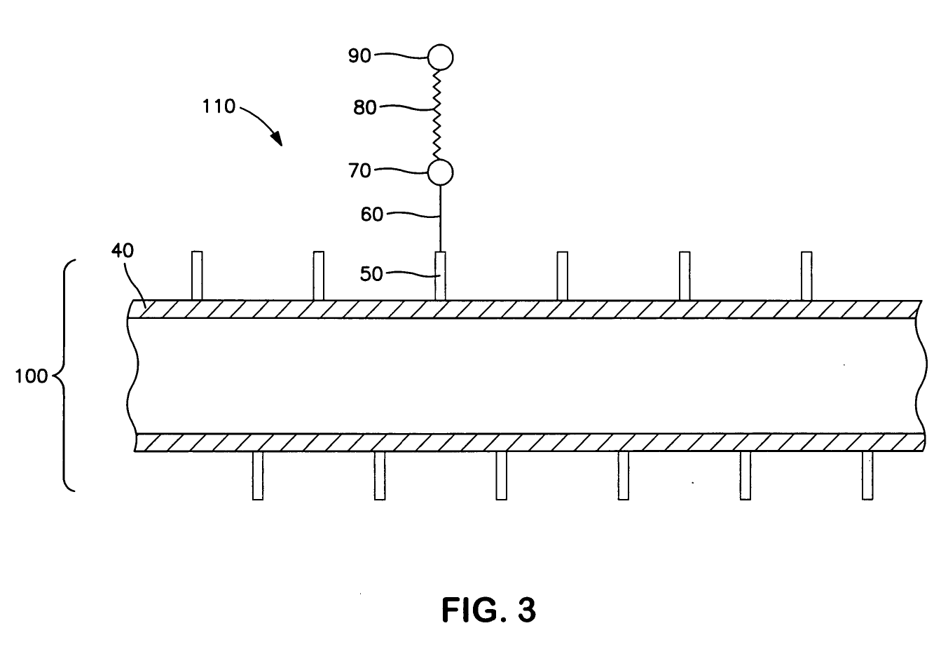

Turning now to FIG. 3, one embodiment of a coated device 110 is shown having a device 100 (e.g., a catheter) with a linker 50 bound or attached to a device wall 40. If the device is formed of a fluorinated polymer, e.g., of PTFE, the linker 50 can be a fluorinated component, to take advantage of an affinity between fluorine atoms of the linker 50 and the fluorinated surface of the device 110.

Bound to the fluorinated component/linker 50 is a carbon-containing component 60. In addition to carbon, the carbon-containing component 60 may contain components other than carbon. Carbon-containing component 60 is disposed between fluorinated component/linker 50 and a head group 70. The head group 70 may be comprised of any compound or atom, however, most typically the head group 70 includes one or more phosphate group(s).

Carbon-containing component 60 and head group 70 may be functionalized phospholipids. One such functionalized phospholipid that can be used is phosphatidylethanolamine. Examples of other functionalized phospholipids that are suitable for use in the present invention include, but are not limited to, N-caproylamine-PE, N-dodecanylamine-PE, phosphatidylthioethanol, and N-MCC-PE. The free amine group on these functionalized phospholipids is free to bind with any other compound and/or component, for example a PEG group. These functionalized phospholipids may also include a fluorine component, to take advantage of the affinity for a fluorine-containing component in the device material, if the device is fabricated of a fluorinated material.

The head group 70 is bound, linked or otherwise adjacent to a PEG group 80. For example, the head group 70 can be covalently bound to the PEG group 80. An active group 90 is bound, linked or otherwise adjacent to PEG group 80. Therefore, the PEG group 80 is disposed between active group 90 and head group 70. The active group 90 may then act via its specific mechanism of action.

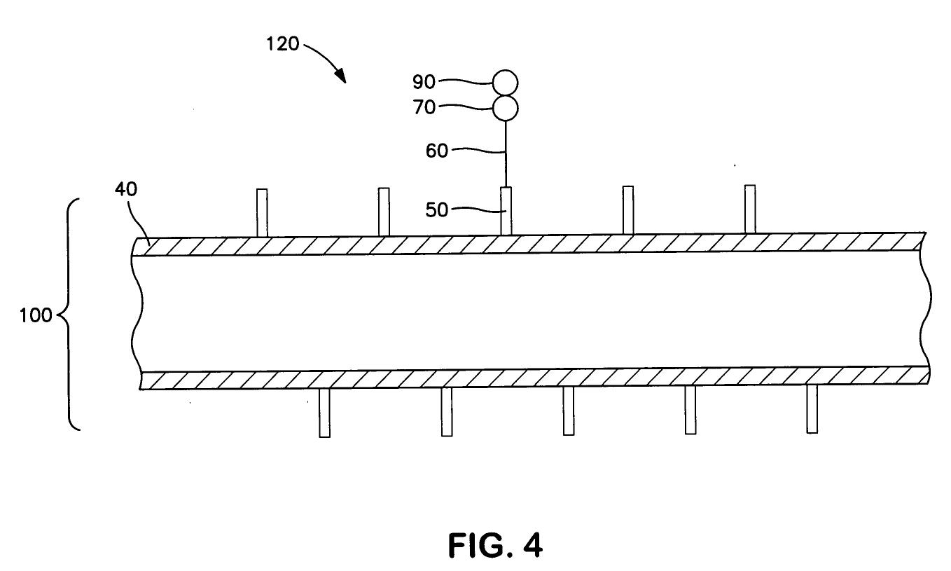

Turning now to FIG. 4, another embodiment of a coated device 120 is shown. Components identified on FIG. 4 by the same number as on FIGS. 1-3, have the same description and characteristics as those described above. In this embodiment, the fluorinated component/linker 50 is bound, attached or otherwise to the wall 40 of device 100 (i.e. a catheter). Bound to the fluorinated component/linker 50 is the carbon-containing component 60.

The carbon-containing component 60 is disposed between the fluorinated component/linker 50 and the head group 70. The head group 70 is bound, linked or otherwise adjacent to active group 90, which may then act via its specific mechanism of action. As can be seen, in this embodiment, the head group 70 is directly adjacent the active component 90 via a bond, linkage or other type of attractiveness. There is no PEG group 80, as described above in the embodiment of FIG. 3.

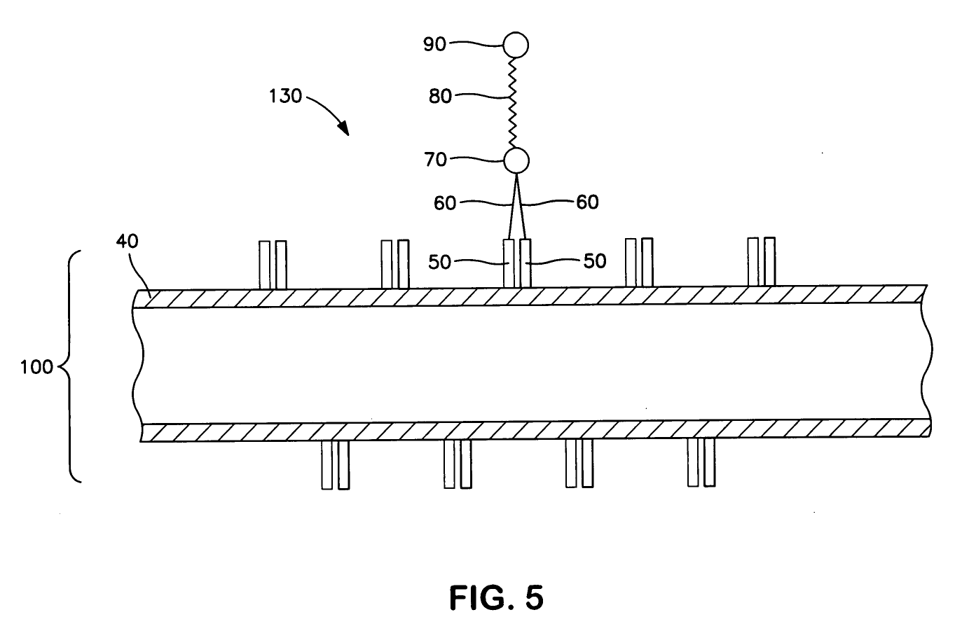

FIG. 5 shows another embodiment of a coated device 130, which is similar to coated device 110 of FIG. 3 except it includes more than one fluorine components/linkers 50 and carbon-containing components 60. In this embodiment, device 100 (i.e. a catheter) has more than one fluorinated component/linker 50 bound or attached to a device wall 40. In this case two fluorine components/linkers are shown but in other embodiments, there may be more than tow. Bound to each fluorinated component/linker 50 is a carbon-containing component 60.

Each carbon-containing component 60 may contain other components than carbon. Each carbon-containing component 60 is dispose between a fluorinated component/linker 50 and the head group 70. The head group 70 is bound, linked or otherwise adjacent to the PEG group 80, and the active group 90 is bound, linked or otherwise adjacent to the PEG group 80. Accordingly, the PEG group 80 is disposed between the active group 90 and the head group 70. The active group 90 may then act via its specific mechanism of action.

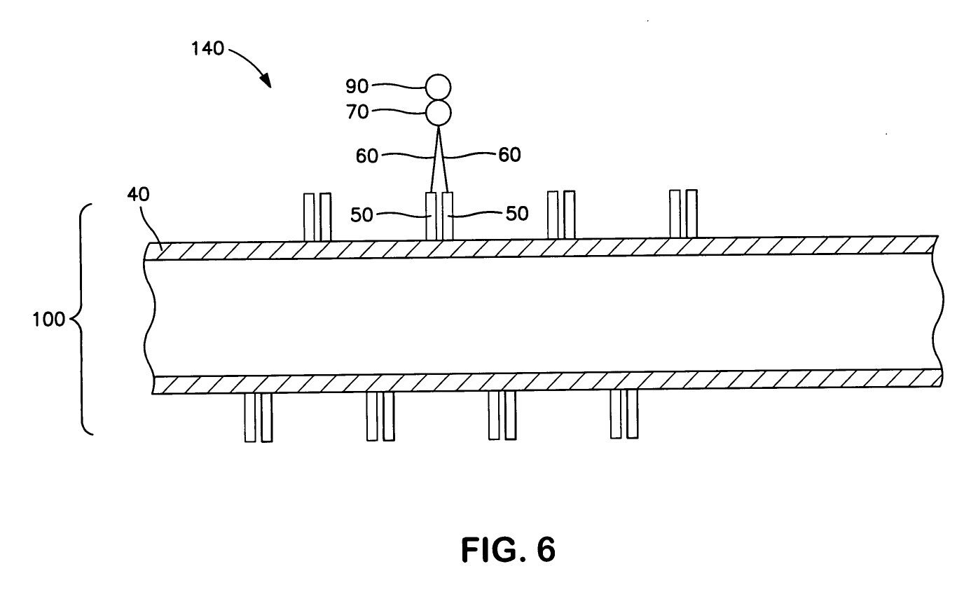

The final embodiment is shown by FIG. 6. A coated device 140, is similar to the coated device 120 of FIG. 4 except it includes more than one fluorine components/linkers 50 and carbon-containing components 60. In this embodiment, the device 100 (e.g., a catheter) has more than one fluorinated component/linker 50 bound or attached to the device wall 40. As an illustration, two fluorine components/linkers are shown but in other embodiments, there may be more than two.

Each carbon-containing component 60 is disposed between a fluorinated component/linker 50 and a head group 70. The head group 70 is bound, linked or otherwise adjacent to active group 90, which may then act via its specific mechanism of action. As can be seen, in this embodiment, the head group 70 is directly adjacent the active component 90 via a bond, linkage or other type of attractiveness. There is no PEG group 80, as described above in the embodiment of FIG. 5.

In one aspect of the invention, in any of the above embodiments there may be a device coating applied to the surface of the device (i.e., catheter). This coating can be applied to the surface of the device that does not include the fluorinated components/linkers, i.e., this coating can be applied to the surface of the device in between the fluorinated components/linkers 50 and the surface 40 of the device 100. This coating may be any coating to prevent unintended non-specific interactions. For example, the coating may be and/or include N,N-bis(trimethylsilyl)acetamide (BSA).

Although the foregoing invention has been described in some detail by way of illustration and example for purposes of clarity and understanding, it will be apparent to those of ordinary skill in the art in light of the teaching of this invention that certain changes and modifications may be made thereto without departing from the spirit or scope of the claims. Such variations and modifications are considered to be within the scope of the invention, which is intended to be limited only to the scope of the claims as interpreted according to the principles of patent law, including the doctrine of equivalents.

What is claimed is:

1. A coated medical device that reduces the incidence of, and/or prevents occlusions from, forming on the device, and/or caused by the presence of the device, the device comprising:
(a) a device having a body;
(b) at least one active component selected from a group consisting of a drug, a peptide, a ligand, a protein, and an antibody; and
(c) at least one bi-functional poly(alkylene glycol) disposed between the body and the active component, wherein the bi-functional poly(alkylene glycol) is bound at one end to the body and bound at the other end to the active component.

2. The coated device of claim 1, wherein the active component is selected from a group consisting of a plasminogen activator, a fibrinolytic enzyme; and a combination thereof.

3. The coated device of claim 1, wherein the active component is a drug selected from the group consisting of a tissue plasminogen activator, a urokinase, a prourokinase and a fibrinolytic enzyme.

4. The coated device of claim 3, wherein the fibrinolytic enzyme is selected from the group consisting of desmoteplase, alfimaprase, and ancrod.

5. The coated device of claim 1, wherein the active component is selected from the group consisting of a peptide, a ligand, a protein and an antibody.

6. The coated device of claim 1, wherein the body is made of a material selected from the group consisting of silicone, polyurethane and fluorinated polymer.

7. The coated device of claim 6, wherein the fluorinated polymer is polytetrafluoroethylene.

8. The coated device of claim 1, wherein the body is coated with polytetrafluoroethylene.

9. The coated device of claim 1, wherein the bi-functional poly(alkylene glycol) is selected from a group consisting of poly(ethylene glycol) and poly(propylene glycol).

10. The coated device of claim 1, wherein the bi-functional poly(alkylene glycol) is poly(ethylene glycol).

11. The coated device of claim 1, wherein the device is selected from the group consisting of a catheter, a stent, a guidewire and a balloon catheter.

12. A coated medical device that reduces the incidence of, and/or prevents occlusions from, forming on the device, and/or caused by the presence of the device, the device comprising:
(a) a device having a body;
(b) at least one carbon-containing component;
(c) at least one fluorinated component disposed between the body and the carbon-containing component;
(d) at least one active group, the active group being a plasminogen activator and/or a fibrinolytic enzyme; and
(e) at least one head group containing one or more phosphate groups disposed between the active group and the carbon-containing component.

13. The coated device of claim 12, wherein the device is selected from the group consisting of a catheter, a stent, a guidewire and a balloon catheter.

14. The coated device of claim 12, wherein the body is made of a material selected from the group consisting of silicone, polyurethane and fluorinated polymer.

15. The coated device of claim 14, wherein the fluorinated polymer is polytetrafluoroethylene.

16. The coated device of claim 12, wherein the body is coated with polytetrafluoroethylene.

17. The coated device of claim 12, wherein the carbon-containing component and head group are functionalized phospholipids selected from the group consisting of phosphatidylethanolamine, N-caproylamine-PE, N-dodecanylamine-PE, phosphatidylthioethanol and N-MCC-PE.
wherein the active component is selected from a group consisting of a plasminogen activator, a fibrinolytic enzyme; and a combination thereof.

18. The coated device of claim 12, wherein the active group comprises a a drug selected from the group consisting of a tissue plasminogen activator, a urokinase, a prourokinase and a fibrinolytic enzyme.

19. The coated device of claim 18, wherein the fibrinolytic enzyme is selected from the group consisting of desmoteplase, alfimaprase, and ancrod.

20. The coated device of claim 12, wherein the active group comprises a component selected from the group consisting of a peptide, a ligand, a protein and an antibody.

21. The coated catheter of claim 12, further comprising at least one bi-functional poly(alkylene glycol) disposed between the head group and the active group.

22. The coated device of claim 21, wherein the bi-functional poly(alkylene glycol) is selected from a group consisting of poly(ethylene glycol) and poly(propylene glycol).

23. The coated device of claim 21, wherein the bi-functional poly(alkylene glycol) is poly(ethylene glycol).

24. The coated device of claim 12, further comprising a coating applied to the body of the device in between the fluorinated components/linkers, wherein the coating includes N,N-Bis(trimethylsilyl)acetamide.
